(19) United States
(12) Patent Application Publication
(10) Pub. No.: US 2007/0298078 A1
(43) Pub. Date: Dec. 27, 2007

(54) Water Soluble Article for Imparting Dietary Fiber to Bottled Water

(76) Inventors: Michael D. Harrison, Decatur, IL (US); Geoffrey A. Nobes, Decatur, IL (US); Andrew J. Hoffman, Mount Zion, IL (US)

Correspondence Address: WILLIAMS, MORGAN & AMERSON, 10333 RICHMOND, SUITE 1100, HOUSTON, TX, 77042, US

(21) Appl. No.: 11/426,638

(22) Filed: Jun. 27, 2006

(51) Int. Cl. A61K 47/00; A61K 9/20; A61K 9/46; A61K 31/715

(52) U.S. Cl. 424/439; 424/464; 514/54; 424/466

(57) ABSTRACT

We disclose a tablet containing at least one water-soluble fiber source, wherein the tablet is of a size and shape susceptible to insertion into a package of a beverage and the tablet will dissolve in the beverage in the absence of agitation in less than about 2 minutes. We also disclose a tablet containing at least one water-soluble fiber source, at least one acidulant, and at least one water-soluble carbonate or bicarbonate, wherein the tablet is of a size and shape susceptible to insertion into a package of a beverage. We also disclose a fiber delivery packet containing at least one water-soluble fiber source and a water-soluble film that encloses the at least one water-soluble fiber source, wherein the packet is of a size and shape susceptible to insertion into a package of a beverage and will dissolve in the beverage in the absence of agitation at a temperature from about 4° C. to about 25° C. in less than about 2 minutes. The tablets and fiber delivery packets we disclose are capable of adding flavor, sweetness, or tartness to bottled water and of allowing consumers to conveniently add fiber to their diets.

### BACKGROUND OF THE INVENTION

The present invention relates generally to the field of fiber fortification of foodstuffs. More particularly, it concerns fiber fortification of beverages by the consumer.

In recent years, bottled water has become a major business around the world. According to Wikipedia, worldwide sales of bottled water are estimated to be between US$50 billion and U.S. $100 billion annually and increasing about 7-10% per annum. In 2004, total worldwide sales were approximately 41 billion gallons (154 billion liters), of which about 6 billion gallons (26 billion liters) were sold in the United States. Although bottled water consumption carries connotations of health, naturalness, and the like, consumer acceptance of bottled water suffers in comparison to that of soft drinks, fruit juices, and sport drinks in that bottled water lacks tartness, sweetness, and flavor and tastes like water. Therefore, a need exists for compositions capable of adding flavor, sweetness, or tartness to bottled water.

Powdery materials, such as sugar free Crystal Light® “On The Go” in tearable paper packs, are commercially available to add flavor, tartness, or sweetness to bottled water, but the packaging of such formulations is susceptible to undesired rips that can spill the powder.

Unrelated to the challenges facing bottled water, described above, is the fact that fiber consumption in the United States and other developed countries is lower than is recommended by nutritional experts. Fiber, by which is meant both insoluble fiber, such as cellulose or related materials, and soluble fiber, by which is meant water-soluble materials indigestible by man, assists in gastrointestinal function and may lower the risk of developing type II diabetes, heart disease, high cholesterol, or obesity. The United States recommended daily value for fiber consumption for a person eating a 2000 calorie/day diet is 25 g, but it is estimated the average United States resident only consumes about 14-15 g per day. Therefore, a need exists for compositions capable of allowing consumers to conveniently add fiber to their diets.

### SUMMARY OF THE INVENTION

In one embodiment, the present invention relates to a tablet containing at least one water-soluble fiber source, wherein the tablet is of a size and shape susceptible to insertion into a package of a beverage and the tablet will dissolve in the beverage in the absence of agitation at a temperature from about 4° C. to about 25° C. in less than about 2 minutes.

In another embodiment, the present invention relates to a tablet containing at least one water-soluble fiber source, at least one acidulant, and at least one water-soluble carbonate or bicarbonate, wherein the tablet is of a size and shape susceptible to insertion into a package of a beverage.

In another embodiment, the present invention relates to a fiber delivery packet containing at least one water-soluble fiber source and a water-soluble film that encloses the at least one water-soluble fiber source, wherein the packet is of a size and shape susceptible to insertion into a package of a beverage and will dissolve in the beverage in the absence of agitation at a temperature from about 4° C. to about 25° C. in less than about 2 minutes

The present invention provides compositions that are capable of adding flavor, sweetness, or tartness to bottled water and of allowing consumers to conveniently add fiber to their diets.

### DESCRIPTION OF ILLUSTRATIVE EMBODIMENTS

In one embodiment, the present invention relates to a tablet containing at least one water-soluble fiber source, wherein the tablet is of a size and shape susceptible to insertion into a package of a beverage and the tablet will dissolve in the beverage in the absence of agitation at a temperature from about 4° C. to about 25° C. in less than about 2 minutes.

Tablets per se and processes and apparatus for making tablets are known to the ordinary skilled artisan. The particular tablets of the present invention can be manufactured as a routine matter for the ordinary skilled artisan having the benefit of the present disclosure. The word “tablet” herein refers to any shaped article formed by pressing of dry components and does not imply any particular size or shape.

In the tablet, each at least one water-soluble fiber source can be selected from the group consisting of pullulan, digestion-resistant maltodextrins, digestion-resistant dextrins, digestion-resistant glucose syrup, inulin, fructooligosaccharides, glucooligosaccharides, beta-glucans, pectins, and guar gum. In one embodiment, the at least one water-soluble fiber source is a digestion-resistant glucose syrup or a digestion-resistant maltodextrin. In another embodiment, the tablet contains at least two water-soluble fiber sources.

In one embodiment, the tablet can contain from about 0.5 g to about 10 g of total water-soluble fiber source (summed over all water-soluble fiber sources present in the tablet). In a further embodiment, the tablet can contain from about 2.5 g to about 5 g of total water-soluble fiber source.

As stated, the tablet is of a size and shape susceptible to insertion into a package of a beverage. Packages of beverages include cans, bottles, cartons, and cups, among others. The package can be a single-serving package, a package not intended as a single serving but generally consumed as such (e.g., 20 fl. oz. to 1 L plastic bottles), or a multi-serving package (e.g., a 2 L plastic bottle). One parameter of the package to be considered when sizing and shaping the tablet is the dimension of the package mouth. For example, a typical 12 fluid ounce (355 mL) single-serving aluminum can with pop-top opening has a roughly oval mouth about 15/16 in (about 2.4 cm) wide and about 7/16 in (about 1.1 cm) between the edge of the tab and the front of the mouth. For another example, a typical 20 fluid ounce (590 mL) plastic bottle with a screw-cap opening has a roughly circular mouth with a nominal diameter of about ¾ in (about 1.9 cm).

By being sized and shaped such that it is susceptible to insertion into a package of a beverage, the tablet can be inserted into the package. As the tablet dissolves, each at least one water-soluble fiber source in the tablet enters solution, therefore fortifying the beverage with dietary fiber and providing dietary fiber to a consumer who drinks the beverage.

Also as stated above, the tablet according to this embodiment of the invention is chosen such that it will dissolve in the beverage in the absence of agitation at a temperature from about 4° C. to about 25° C. in less than about 2 minutes. “Absence of agitation” means the state of allowing the package of the beverage to stand on a substantially fixed surface (e.g., a tabletop, a cupholder of an automobile at cruising speed traveling in a substantially straight path, etc). The temperature range refers to the temperature of the beverage in the package at the time of insertion of the tablet. The reader must not construe this discussion as limiting the use of the tablet to beverages that are not agitated and are within the recited temperature range. Rather, this discussion solely establishes a set of testing parameters for determining the time to dissolution of the tablet. The skilled artisan having the benefit of the present disclosure can readily picture a consumer inserting the tablet into a screw-cap bottle, recapping the bottle, and shaking the bottle to accelerate dissolution of the tablet or a consumer inserting the tablet into a cup of hot coffee or hot tea. We have clearly contemplated such uses for the tablet of the present invention.

As used herein, “beverage” refers to any potable aqueous solution. Any beverage for which fiber fortification may be desired can be used. In one embodiment, the beverage is selected from the group consisting of water, milk, fruit juices, vegetable juices, carbonated soft drinks, non-carbonated soft drinks, coffee (iced or hot, and including coffee-based beverages such as cappuccinos, lattes, mochas, iced blended coffee drinks), tea (iced or hot), beer, wine, liquor, and alcoholic mixed drinks.

The tablet contains at least one water-soluble fiber source. It may also contain one or more components other than water-soluble fiber sources.

In one embodiment, the tablet further contains at least one acidulant. An acidulant is a material acceptable for human or animal consumption that lowers the pH of an aqueous solution into which it is dissolved. In one embodiment, the acidulant can be selected from the group consisting of citric acid and malic acid. The inclusion of at least one acidulant in the tablet can impart tartness to the beverage in which the tablet will be dissolved.

In one embodiment, the tablet further contains at least one water-soluble carbonate or bicarbonate. On entering an aqueous solution, the water-soluble carbonate or bicarbonate imparts carbonation to the aqueous solution. The water-soluble carbonate or bicarbonate should be acceptable for human or animal consumption. In one embodiment, each at least one water-soluble carbonate or bicarbonate can be selected from the group consisting of sodium carbonate and calcium carbonate. In a further embodiment, the at least one water-soluble carbonate or bicarbonate can be sodium carbonate. The inclusion of at least one water-soluble carbonate or bicarbonate in the tablet can impart fizz to the beverage in which the tablet will be dissolved.

In another embodiment, the tablet further contains at least one flavorant. A flavorant is a material acceptable for human or animal consumption that imparts a flavor to an aqueous solution into which it is dissolved. In one embodiment, each at least one flavorant is selected from the group consisting of lemon flavor, lime flavor, cherry flavor, strawberry flavor, banana flavor, blueberry flavor, grape flavor, watermelon flavor, orange flavor, apple flavor, peach flavor, raspberry flavor, chocolate flavor, vanilla flavor, bubble gum flavor, and licorice flavor.

In another embodiment, the tablet further contains at least one sweetener. A sweetener is a nutritive or non-nutritive material acceptable for human or animal consumption that imparts a sweet taste to an aqueous solution into which it is dissolved. In one embodiment, each at least one sweetener is selected from the group consisting of sucralose, saccharin, aspartame, and acesulfame salts. The most commonly used acesulfame salt in the food industry in the United States at this writing is acesulfame potassium.

In another embodiment, the tablet further contains at least one colorant. A colorant is a material acceptable for human or animal consumption that imparts a color to an aqueous solution into which it is dissolved.

In another embodiment, the tablet further contains at least one preservative. A preservative is a material acceptable for human or animal consumption that protects other materials from attack by microbes, insects, or other pests.

Two or more of the further components listed above can be included in the tablet. For example, inclusion of citric acid, lemon flavor, and a sweetener in the tablet can impart a lemonade profile to the beverage into which the tablet is dissolved.

In another embodiment, the present invention relates to a tablet containing at least one water-soluble fiber source, at least one acidulant, and at least one water-soluble carbonate or bicarbonate, wherein the tablet is of a size and shape susceptible to insertion into a package of a beverage.

Each at least one water-soluble fiber source can be as described above.

Each at least one acidulant can be as described above. In one embodiment, the tablet contains from about 0.1 g to about 1 g of total acidulant (summed over all acidulants in the tablet). In a further embodiment, the tablet contains from about 0.25 g to about 0.75 g of total acidulant.

Each at least one water-soluble carbonate or bicarbonate can be as described above. In one embodiment, the tablet contains from about 0.1 g to about 1 g of total water-soluble carbonate or bicarbonate (summed over all water-soluble carbonates or bicarbonates in the tablet). In a further embodiment, the tablet contains from about 0.25 g to about 0.75 g of total water-soluble carbonate or bicarbonate.

By containing both at least one acidulant and at least one water-soluble carbonate or bicarbonate, the tablet both lowers the pH of and imparts fizz to the beverage, which can aid in the dissolution of the tablet and can fiber-fortify the beverage more rapidly than would be the case in the absence of the at least one acidulant and at least one water-soluble carbonate or bicarbonate. In one embodiment, the inclusion of the at least one acidulant and the at least one water-soluble carbonate or bicarbonate in the tablet can allow the tablet to dissolve in the beverage in the absence of agitation and at a temperature between about 4° C. and about 25° C. in less than about 90 seconds.

The beverage can be as described above.

The tablet of this embodiment can further contain one or more of the materials described above. In one embodiment, the tablet can further contain at least one flavorant, as described above. In one embodiment, the tablet can further contain at least one sweetener, as described above.

In another embodiment, the present invention relates to a fiber delivery packet containing at least one water-soluble fiber source and a water-soluble film that encloses the at least one water-soluble fiber source, wherein the packet is of a size and shape susceptible to insertion into a package of a beverage and will dissolve in the beverage in the absence of agitation at a temperature from about 4° C. to about 25° C. in less than about 2 minutes.

Each at least one water-soluble fiber source can be as described above.

A number of water-soluble films are known in the art. In one embodiment, the film contains a major amount of pullulan on a dry solids basis, and a minor amount of more than one member selected from glycerol, propylene glycol, and sorbitol. The water-soluble film of this particular embodiment can be as described in U.S. patent application Ser. No. 11/424,586, the disclosure of which is hereby incorporated by reference.

The beverage can be as described above.

The fiber delivery packet of this embodiment can further contain one or more of the materials described above. Each material further contained in the fiber delivery packet can be enclosed within the water-soluble film, incorporated into the water-soluble film, or both. In one embodiment, the fiber delivery packet can further contain at least one acidulant, as described above. In one embodiment, the fiber delivery packet can further contain at least one water-soluble carbonate or bicarbonate, as described above. In one embodiment, the fiber delivery packet can further contain at least one flavorant, as described above. In one embodiment, the fiber delivery packet can further contain at least one sweetener, as described above.

A tablet or fiber delivery packet according to any embodiment of the present invention can be used in a method of fortifying a beverage with fiber, comprising inserting the tablet into a package containing the beverage.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Four grams of digestion resistant maltodextrin (Fibersol-2®, Matsutani) was dry blended with 0.5 grams of citric acid and 0.5 grams of sodium carbonate until well mixed. Small portions of the dry powder were filled into a metal cylinder capable of compressing the material into a small tablet shape of about ⅝ inch diameter and about ¼ inch thickness. All of the dry material was pressed into a total of four tablets weighing about 1.25 grams each. The tablets were then added to a freshly opened 20 fl. oz. bottle of Sam's Choice™ Purified Drinking Water. The tablets immediately began to dissolve with bubbling action. After about 1 minute the tablets were completely dissolved, leaving a clear solution in the bottle that was slightly salty to the taste. Complete consumption of the drink would deliver about 3.6 grams of dietary fiber, or about 15% of the recommended daily value for a person eating a 2000 calorie/diet in the United States or Canada.

### EXAMPLE 2

Four grams of digestion resistant maltodextrin (Fibersol-2®, Matsutani) was dry blended with 0.5 grams of citric acid and 0.5 grams of sodium carbonate and 1 gram of Lemonade Dry Mix (see below) until well mixed.

Lemonade Dry Mix

| | |
|---|---|
| Citric Acid | 0.394 grams |
| Malic Acid | 0.209 grams |
| Splenda Dry powder | 0.046 grams |
| Yellow #5 | 0.002 grams |
| Nat. & Art. Lemon Flavor #SD10059 | 0.696 grams |

Small portions of the dry powder were filled into a metal cylinder capable of compressing the material into a small tablet shape of about ⅝ inch diameter and about ¼ inch thickness. All of the dry material was pressed into a total of four tablets weighing about 1.5 grams each. The tablets were then added to a freshly opened 20 fl. oz. bottle of Sam's Choice™ Purified Drinking Water. The tablets immediately began to dissolve with bubbling action. After about 1 minute the tablets were completely dissolved, leaving a clear yellow solution in the bottle that had a delicious lemonade taste. Complete consumption of the drink would deliver about 3.6 grams of dietary fiber, or about 15% of the recommended daily value for a person eating a 2000 calorie/day diet in the United States or Canada.

All of the compositions disclosed and claimed herein can be made and executed without undue experimentation in light of the present disclosure. While the compositions of this invention have been described in terms of preferred embodiments, it will be apparent to those of skill in the art that variations may be applied to the compositions described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A tablet, comprising:
at least one water-soluble fiber source,
wherein the tablet is of a size and shape susceptible to insertion into a package of a beverage and the tablet will dissolve in the beverage in the absence of agitation at a temperature from about 4° C. to about 25° C. in less than about 2 minutes.

2. The tablet of claim 1, wherein each at least one water-soluble fiber source is selected from the group consisting of pullulan, digestion-resistant maltodextrins, digestion-resistant dextrins, digestion-resistant glucose syrup, inulin, fructooligosaccharides, glucooligosaccharides, beta-glucans, pectins, and guar gum.

3. The tablet of claim 2, comprising at least two water-soluble fiber sources.

4. The tablet of claim 2, wherein the at least one water-soluble fiber source is a digestion-resistant glucose syrup or a digestion-resistant maltodextrin.

5. The tablet of claim 1, wherein the beverage is selected from the group consisting of water, milk, fruit juices, vegetable juices, carbonated soft drinks, non-carbonated soft drinks, coffee, tea, beer, wine, liquor, and alcoholic mixed drinks.

6. A tablet, comprising:
at least one water-soluble fiber source,
at least one acidulant, and
at least one water-soluble carbonate or bicarbonate,
wherein the tablet is of a size and shape susceptible to insertion into a package of a beverage.

7. The tablet of claim 6, wherein the tablet will dissolve in the beverage in the absence of agitation at a temperature from about 4° C. to about 25° C. in less than about 90 seconds.

8. The tablet of claim 6, wherein each at least one water-soluble fiber source is selected from the group consisting of pullulan, digestion-resistant maltodextrins, digestion-resistant dextrins, digestion-resistant glucose syrup, inulin, fructooligosaccharides, glucooligosaccharides, beta-glucans, pectins, and guar gum.

9. The tablet of claim 8, wherein the tablet comprises at least two water-soluble fiber sources.

10. The tablet of claim 8, wherein the at least one water-soluble fiber source is a digestion-resistant glucose syrup or a digestion-resistant maltodextrin.

11. The tablet of claim 6, wherein the beverage is selected from the group consisting of water, milk, fruit juices, vegetable juices, carbonated soft drinks, non-carbonated soft drinks, coffee, tea, beer, wine, liquor, and alcoholic mixed drinks.

12. The tablet of claim 6, wherein each at least one acidulant is selected from the group consisting of citric acid and malic acid.

13. The tablet of claim 6, wherein each at least one water-soluble carbonate or bicarbonate is selected from the group consisting of sodium carbonate and calcium carbonate.

14. The tablet of claim 6, further comprising:
at least one flavorant.

15. The tablet of claim 14, wherein each at least one flavorant is selected from the group consisting of lemon flavor, lime flavor, cherry flavor, strawberry flavor, banana flavor, blueberry flavor, grape flavor, watermelon flavor, orange flavor, apple flavor, peach flavor, raspberry flavor, chocolate flavor, vanilla flavor, bubble gum flavor, and licorice flavor.

16. The tablet of claim 6, further comprising at least one sweetener.

17. The tablet of claim 16, wherein each at least one sweetener is selected from the group consisting of sucralose, saccharin, aspartame, and acesulfame salts.

18. The tablet of claim 6, comprising from about 0.5 g to about 10 g of total water-soluble fiber source.

19. The tablet of claim 6, comprising from about 0.1 g to about 1 g of total acidulant.

20. The tablet of claim 6, comprising from about 0.1 g to about 1 g of total water-soluble carbonate or bicarbonate.

21. A fiber delivery packet, comprising:
at least one water-soluble fiber source, and
a water-soluble film that encloses the at least one water-soluble fiber source,
wherein the packet is of a size and shape susceptible to insertion into a package of a beverage and will dissolve in the beverage in the absence of agitation at a temperature from about 4° C. to about 25° C. in less than about 2 minutes.

22. The fiber delivery packet of claim 21, wherein the film contains a major amount of pullulan on a dry solids basis, and a minor amount of more than one member selected from glycerol, propylene glycol, and sorbitol.

23. The fiber delivery packet of claim 21, wherein each at least one water-soluble fiber source is selected from the group consisting of pullulan, digestion-resistant maltodextrins, digestion-resistant dextrins, digestion-resistant glucose syrup, inulin, fructooligosaccharides, glucooligosaccharides, beta-glucans, pectins, and guar gum.

24. The fiber delivery packet of claim 23, comprising at least two water-soluble fiber sources.

25. The fiber delivery packet of claim 23, wherein the at least one water-soluble fiber source is a digestion-resistant glucose syrup or a digestion-resistant maltodextrin.

26. The fiber delivery packet of claim 21, wherein the beverage is selected from the group consisting of water, milk, fruit juices, vegetable juices, carbonated soft drinks, non-carbonated soft drinks, coffee, tea, beer, wine, liquor, and alcoholic mixed drinks.

27. The fiber delivery packet of claim 21, further comprising at least one acidulant.

28. The fiber delivery packet of claim 27, wherein each at least one acidulant is selected from the group consisting of citric acid and malic acid.

29. The fiber delivery packet of claim 21, further comprising at least one water-soluble carbonate or bicarbonate.

30. The fiber delivery packet of claim 29, wherein each at least one water-soluble carbonate or bicarbonate is selected from the group consisting of sodium carbonate and calcium carbonate.

31. The fiber delivery packet of claim 21, further comprising at least one flavorant.

32. The fiber delivery packet of claim 31, wherein each at least one flavorant is selected from the group consisting of lemon flavor, lime flavor, cherry flavor, strawberry flavor, banana flavor, blueberry flavor, grape flavor, watermelon flavor, orange flavor, apple flavor, peach flavor, raspberry flavor, chocolate flavor, vanilla flavor, bubble gum flavor, and licorice flavor.

33. The fiber delivery packet of claim 21, further comprising at least one sweetener.

34. The fiber delivery packet of claim 33, wherein each at least one sweetener is selected from the group consisting of sucralose, saccharin, aspartame, and acesulfame salts.

35. A method of fortifying a beverage with fiber, comprising:
inserting into a package containing the beverage a tablet comprising at least one water-soluble fiber source, at least one acidulant, and at least one water-soluble carbonate or bicarbonate, wherein the tablet is of a size and shape susceptible to insertion into a package of a beverage, or a fiber delivery packet, comprising at least one water-soluble fiber source and a water-soluble film that encloses the at least one water-soluble fiber source, wherein the film contains a major amount of pullulan on a dry solids basis, and a minor amount of more than one member selected from glycerol, propylene glycol, and sorbitol and the packet is of a size and shape susceptible to insertion into a package of a beverage and will dissolve in the beverage in the absence of agitation at a temperature from about 4° C. to about 25° C. in less than about 2 minutes.
